(19) United States
(12) Patent Application Publication
(10) Pub. No.: US 2004/0014184 A1
(43) Pub. Date: Jan. 22, 2004

(54) Method for obtaining 12-hydroxystearic acid

(76) Inventors: Ralf Otto, Bad Friedrichshall (DE); Ulrich Schoerken, Leichlingen (DE); Albrecht Weiss, Langenfeld (DE); Levent Yueksel, Duesseldorf (DE); Georg Fieg, Mettmann (DE); Sabine Both, Duesseldorf (DE); Sabine Kranz, Duesseldorf (DE)

Correspondence Address: COGNIS CORPORATION, PATENT DEPARTMENT, 300 BROOKSIDE AVENUE, AMBLER, PA, 19002, US

(21) Appl. No.: 10/415,784

(22) PCT Filed: Oct. 25, 2001
(86) PCT No.: PCT/EP01/12360

(30) Foreign Application Priority Data
Nov. 3, 2000 (DE) 100-54-480.0

(51) Int. Cl. C12P 7/64; C07C 51/36

(52) U.S. Cl. 435/134; 554/141

(57) ABSTRACT

Disclosed is a method for obtaining 12-hydroxystearinic acid and the salts thereof from a native fat or oil, especially ricinoleic oil, characterized in that a) the native fat or oil is hydrolized under the catalytic influence of one or several enzymes at 15-50° C. to obtain ricinoleic acid b) the glycerol thus arising and the enzyme are separated, c) the hydrolysate is catalytically hydrolized, d) the product thus obtained is formulated.

### FIELD OF THE INVENTION

This invention relates generally to the isolation of 12-hydroxystearic acid and, more particularly, to a process for the isolation of 12-hydroxystearic acid from a native fat or oil, more particularly from castor oil.

### PRIOR ART

12-Hydroxystearic acid is a C₁₈ fatty acid which is derived from ricinoleic acid and which has the chemical empirical formula C₁₈H₃₆O₃. It consists of crystals that are colorless at room temperature. 12-Hydroxystearic acid is used in the form of its salts, the 12-hydroxystearates, as an intermediate stage in the production of plastics or as an ingredient of cosmetic products.

The isolation and production of 12-hydroxystearic acid from oil is already known and has already been widely described in the literature/patent literature. Hitherto, castor oil has been described as a starting material for the isolation of 12-hydroxystearic acid. Depending on its origin, crude castor oil contains between 87 and 91% ricinoleic acid in the form of the glycerides, 2% stearic and palmitic acid, 4-5% oleic acid and 4-5% linoleic acid. The acids are present in the form of their glycerides. Castor oil is obtained by cold pressing of the seeds of the castor-oil plant, Rizinus communes.

The reaction conditions of a conventional lipolysis process cannot be applied to the isolation of 12-hydroxystearic acid because ricinoleic acid and 12-hydroxystearic acids are destroyed under those conditions.

In conventional processes for isolating 12-hydroxystearic acid, the castor oil is first chemically hydrogenated in a heterogeneous metal-catalyzed reaction, the hydrogenated oil is then chemically split by alkaline ester cleavage and is subsequently neutralized by acid so that 12-hydroxystearic acid is obtained.

In Rev. Soc. Quim. Mex. 37, 1993, pp. 66-69, C. Melanco describes how castor oil is hydrogenated in the presence of an Ni and Pd catalyst and how this hydrogenated castor oil is split by alkaline saponification to give 12-hydrostearic acid. The yields of 12-hydroxystearic acid obtained in this process are very poor.

In an article in JAOCS, 65 (9) 1988, 1467-1469, R. K. Trivedi describes the hydrogenation of castor oil by a process in which the hydrogenation is carried out under a hydrogen pressure of 2 bar, at a temperature of 130° C. and in the presence of 2% by weight of an Ni catalyst. Unfortunately, this process for the hydrogenation of castor oil gives a very poor yield. In addition, there is no indication of how the free 12-hydroxystearic acid can be isolated from the hydrogenated oil.

GB 1,130,092 from 1966 describes a process for the hydrogenation of castor oil. The castor oil is hydrogenated at temperatures of up to 180° C. in the presence of an Ni catalyst. In this process, however, the 12-hydroxystearic acid is not released and isolated, instead the hydroxy group of the fatty acids in the hydrogenated castor oil is dehydrated.

In the above-mentioned processes for the chemical production of 12-hydroxystearic acid from castor oil by direct hydrogenation of the castor oil and subsequent saponification of the hydrogenated oil, not only high reaction temperatures but also large quantities of metal catalyst are required. The yields of hydrogenated oil and 12-hydroxystearic acid are sometimes very poor. Besides the high reaction temperatures, the large quantity of catalyst and the poor yield, disadvantages of these conventional processes also include the high salt content of the wastewater after the alkaline saponification and the formation of secondary products. These secondary products are above all dimers and polymers of hydroxystearic acid which can be formed where the saponification is carried out under the above-mentioned conditions. The removal of these secondary products involves another reaction step and is by no means a formality in view of the similar chemical properties of the products.

In another process for isolating 12-hydroxystearic acid known from the prior art (JP 61139396), hydrogenated castor oil is subjected to careful enzymatic hydrolysis. According to the abstract of the patent specification, hydrogenated castor oil is hydrolyzed at 75° C. in the presence of a lipase from a microorganism of various geni. In addition, at that temperature, the hydrolysis has to be carried out in the presence of a solvent because hydrogenated castor oil has a melting point above 75° C. With the lipases used, the degree of hydrolysis is 84%. There is no indication of how the castor oil is hydrogenated. The disadvantage of this process is the low degree of hydrolysis of only 84% and the high reaction temperature. The high temperature precludes the use of various temperature-sensitive lipases. Another disadvantage common to all processes starting out from hydrogenated castor oil is that the intermediate product, ricinoleic acid, cannot be isolated. Besides 12-hydroxystearic acid, ricinoleic acid is also of considerable interest for many applications and is accessible to only a limited extent by conventional processes.

Several processes for the enzymatic hydrolysis of fats and oils, particularly castor oil, are known from the prior art. For example, the abstract of JP 01016592 describes a lipase-catalyzed process for the hydrolysis of castor oil under mild conditions in which a degree of hydrolysis of more than 70% is achieved. However, there is no indication of how high the degree of hydrolysis really is. In addition, a disadvantage of this process is the large quantity of enzyme used which can amount to between 0.15 and 15% by weight, based on the total quantity of oil used. Where 10 to 15% by weight enzyme is used as catalyst, this process becomes ineffective and very cost-intensive. In addition, it is not apparent to the expert from the broadly worded abstract what quantity of catalyst it is that produces the required degree of hydrolysis.

In the processes for the enzymatic hydrolysis of castor oil, particularly in the cited patent specification, there is no indication of how the free ricinoleic acid can be isolated from the hydrolyzed oil or, more particularly, how the 12-hydroxystearic acid can be isolated from it in high yields.

The lipase-catalyzed hydrolysis of castor oil is also known from the scientific literature. However, the processes described there cannot be scaled up for industrial application. Thus, the use of a lipase from a pathogenic organism (Pseudomonas aeruginosa) is described (Sharon et al., Indian. J. Expl. Biol., 1999, 37, 481 et seq). In addition, lipases from pig's pancreas are also used (Biosci. Biotechnol. Biochem., 1992, 56, 1490 et seq) which would result in a loss of the “kosher” certification of the unit and the secondary product glycerol. Work on the use of plant lipases has shown that only low degrees of hydrolysis of the castor oil are achieved (Fuchs et al., J. Plant Physiol., 1996, 149, 23). These lipases belong to the group of “acidic” lipases, i.e. complicated pH adjustment and buffering of the water phase would be necessary.

### DESCRIPTION OF THE INVENTION

The problem addressed by the present invention was to provide an industrial process involving only a few steps for the effective and economic production of 12-hydroxystearic acid in high yields and purity from a native fat or oil where toxicologically and ecologically unsafe reaction steps would largely be avoided. Another problem addressed by the invention was to provide a process for isolating 12-hydroxystearic acid in which ricinoleic acid would be obtainable as an intermediate product.

The present invention relates to a process for isolating 12-hydroxystearic acid and salts thereof from a native fat or oil, more particularly from castor oil, characterized in that

a) in a first step, the native fat or oil is hydrolyzed at a temperature of 15 to 50° C. in the presence of one or more enzymes as catalyst, ricinoleic acid being formed,

b) the glycerol formed and the enzyme are removed,

c) the hydrolyzate is catalytically hydrogenated,

d) the product thus obtained is made up into an end product.

It has surprisingly been found that the hydrolysis of castor oil in the presence as catalyst of an enzyme or preferably a combination of several enzymes, preferably two enzymes, gives a mixture which, after the removal of enzymes and the glycerol formed, contains a high percentage of free ricinoleic acid which can be hydrogenated under mild conditions and thus gives 12-hydroxystearic acid in highly pure form.

Accordingly, the sequence of reaction steps is critical to the invention. The enzymatic hydrolysis of the native fat or oil has to be carried out first and is followed—after removal of the catalyst and the glycerol formed—by hydrogenation of the products obtained in which the ricinoleic acid can be hydrogenated to form 12-hydroxystearic acid. This process leads to a largely odorless and colorless product.

A native fat or oil in the context of the present invention is understood to be any fat or oil which has a castor oil glyceride content of more than 50%. More particularly, the native fat or oil is castor oil.

Salts of 12-hydroxystearic acid in the context of the invention are understood to be the melt salts, more particularly the alkali metal and alkaline earth metal salts.

Reaction step a)

The reaction conditions according to the invention in reaction step a) of the enzymatic catalysis are determined by the optimal reaction range of the enzymes selected. More particularly, the reaction conditions are inter alia a reaction temperature of 15 to 50° C., preferably in the range from 20 to 40° C. and, more particularly, 35° C.

In another embodiment of the invention, the enzymatic catalysts to be used in step a) are selected from the group of hydrolases, especially the ester hydrolases, which are also known as lipases. According to the invention, the preferred lipases are lipases from Aspergillus oryzae, Aspergillus niger, Bacillus species, Penicillium, camembertii, Pseudomonas cepacia, Candida lipolytica, Geotrichum candidum, Penicillium roqueforti, Rhizopus arrhizus, Rhizopus oryzae, Rhizopus niveus, Mucor javanicus, Rhizomucor miehei and Thermomyces lanugenosus, more particularly the lipase from Aspergillus oryzae or Thermomyces lanugenosus. Aspergillus oryzae, Bacillus species Rhizopus arrhizus or Thermomyces lanugenosus are particularly preferred.

The lipases to be used in accordance with the invention may be used on their own or in combination with several enzymes, a combination of two enzymes being particularly preferred. Such combinations preferably consist of lipases where, on the one hand, the lipases particularly catalyze the 1,3-specific cleavage of glycerides (such lipases are also known as 1,3-specific lipases) and other lipases which specifically catalyze the cleavage of mono(2)-glycerides. The choice can be optimized in each individual case so that, in the best case, none of the lipases used forms unwanted secondary products of ricinoleic acid (dimers or lactones) through transesterification.

The lipases from Thermomyces lanugenosus or Aspergillus oryzae or Rhizopus arrhizus are preferably combined with monoglyceride-specific Penicillium camembertii or Bacillus species lipase. In a particularly preferred embodiment, the lipases from Thermomyces lanugenosus are preferably used with Penicillium camembertii lipase.

The enzymes to be used in accordance with the invention may be used in various forms. In principle, any supply form of enzymes familiar to the expert may be used. The enzymes are preferably used in pure form or as a technical enzyme preparation either immobilized and/or in solution, more particularly aqueous solution.

In another embodiment of the invention, the enzymes to be used in accordance with the invention are used in a quantity of 0.002 to 0.505% by weight, based on the total quantity of native oil or fat used. In one particular embodiment, the quantity used is in the range from 0.002 to 0.140% by weight, a quantity of 0.0520 to 0.1004% by weight being particularly preferred.

Where a technical enzyme preparation is used, the use of 0.004 to 0.5% by weight of an aqueous solution, based on the total quantity of native fat or oil used, is preferred. The use of 0.004 to 0.02% by weight of an aqueous solution of Penicillium camemberti and/or 0.1 to 0.5% by weight of an aqueous solution of Thermomyces lanugenosus is particularly preferred. The percentage of active enzyme in the particular technical enzyme preparations varies from manufacturer to manufacturer. However, the average is 10% active enzyme.

Suitable buffers may optionally be used as other reaction components. Buffers suitable for the purposes of the invention are those which are capable of buffering off a lipase-catalyzed lipolysis process. These buffers are systems which should not destroy the catalyst lipase or impair its activity. Such buffers include, for example, the phosphate buffer or the carbonate buffer. The phosphate buffer is particularly preferred. In a preferred embodiment, the buffer to be used in accordance with the invention is used in a quantity of 0.01 to 0.2% by weight, based on the total quantity of native fat or oil, a quantity of 0.01 to 0.05% by weight being particularly preferred. In a particularly preferred embodiment, however, the lipolysis is carried out in an unbuffered system.

The degree of hydrolysis under the conditions mentioned above is between 90 and 98%.

Reaction step b)

In a second reaction step, the glycerol formed during the hydrolysis has to be removed. In addition, the enzyme catalyst used has to be removed. In principle, the glycerol and the enzyme catalysts used may be removed by any known separation process by which the compounds mentioned and catalysts can be removed, separation by heating of the reaction mixture to 70° C.-90° C. being preferred. Removal by phase separation is particularly preferred. Phase separation is carried out by gravity and the difference in density of the hydrolyzate mixture. In one possible embodiment, the separation process is centrifuging which is preferably carried out continuously for 6 hours at 800 revolutions per minute and under a pressure of 1.2 to 1.3 bar.

According to the invention, reaction step a) and reaction step b) may be repeated several times, depending on the required degree of hydrolysis, before the hydrolyzate is hydrogenated. A single repetition is preferred. This leads under the conditions mentioned to a degree of hydrolysis of 99.5 to 100%.

Reaction step c)

The hydrolyzate obtained after reaction steps a) and b) consists largely of ricinoleic acid. The ricinoleic acid content is dependent on the quality of the castor oil used and on the degree of hydrolysis. The castor oil obtained is hydrogenated in a following reaction step to obtain the 12-hydroxystearic acid. Basically, any hydrogenation catalyst may be used as the catalyst for hydrogenation of the ricinoleic acid.

In principle, two types of catalysis may be used in the hydrogenation according to the invention. In the case of heterogeneous catalysis, a catalyst insoluble in the reaction medium is present and it is on the surface of that catalyst that the actual catalysis is effected through the adsorption and desorption equilibrium of the compound to be hydrogenated. The catalysts used are noble metals, such as for example Pt, Pd or Rh, or other transition metals, such as Mo, W, Cr. Fe, Co and Ni either individually or in admixture are preferred. To increase activity and selectivity, the catalysts may be applied to supports, such as active carbon, aluminium oxide or kieselguhr. Ni or Raney nickel, Pd fixed to active carbon, metallic Pt, platinum and zinc oxide are preferably used in accordance with the invention.

The homogeneous catalysts, i.e. catalysts soluble in the reaction medium, are transition metal complexes of which the preferred representative is the Wilkinson catalyst [chlorotris(triphenyl-phosphine) rhodium].

In a preferred embodiment, the catalysts are heterogeneous catalysts. An Ni catalyst or a Pd catalyst, the Pd being adsorbed onto active carbon, is particularly preferred.

In one particular embodiment, the hydrogenation according to the invention is carried out at a temperature of 70 to 150° C., preferably at a temperature of 90 to 130° C. and more particularly at a temperature of 120° C.

In another preferred embodiment, the hydrogenation is carried out under a pressure of the hydrogen gas of 1 to 300 bar, preferably 5 to 50 bar and, more particularly, 20 bar.

In another preferred embodiment, the hydrogenation catalyst is used in a quantity of 0.2 to 5% by weight, based on the total quantity of native fat or oil used, a quantity of 0.4 to 2% by weight being particularly preferred.

Reaction step d)

In a final process step, the product obtained is made up into an end product without any further treatment or processing. This is preferably done by spray drying although, in principle, it may also be done by any other method for making up solids capable of being melted such as, for example, processing in cutting and shearing mills, granulators, pelleting rollers and flake-forming rollers.

The product obtained, 12-hydroxystearic acid, is largely odorless and largely colorless which could not be achieved to the same extent by known methods. In addition, the product obtained is substantially free from secondary products, such as mono-, di- or triglycerides.

The present invention includes the observation that, through the sequence of the process steps and the combination of an enzymatic and a chemical catalysis, an economic and ecologically safe process has been developed for the production of high-purity ricinoleic acid and 12-hydroxystearic acid from castor oil.

The ricinoleic acid obtained by the process according to the invention and the 12-hydroxystearic acid obtained are suitable for use in cosmetic and pharmaceutical preparations, in lubricants, in textile auxiliaries and for the production of plastics.

The invention is illustrated by the following Examples.

### EXAMPLES

### Example 1

### Screening of Various Lipases for their Hydrolysis Activity with Castor Oil as Substrate

5 g castor oil and 5 g distilled water were stirred at 25° C. to form an emulsion. Various lipases were added in quantities of 5% by weight, based on the oil, and the mixtures were stirred for 96 h at 25° C. Samples were analyzed after 24, 48 and 72 h. The emulsion was separated by centrifuging (5 mins., 13,000 r.p.m.) and the oil phase was analyzed for cleavage products by thin-layer chromatography.

TABLE 1
Hydrolysis activity of various lipases

| | Oil | |
|---|---|---|
| Lipase (origin) | hydrolysis | Remarks |
| Aspergillus oryzae | ++++ | No secondary products |
| | | Monoglyceride accumulation |
| | | (24 h) |
| | | Degree of hydrolysis |
| | | >85% after 48 h |
| Aspergillus niger | + | |
| Burholderia cepacia | + | |
| (formerly: Pseudomonas | | |
| cepacia) | | |
| Candida lipolytica | + | |
| Candida rugosa (formerly: | ++ | Secondary products |
| Candida cylindracea) | | |
| Candida antarctica | o | Secondary products |
| Mucor javanicus | ++ | No secondary products |
| | | Monoglyceride accumulation |
| | | (24 h) |
| (Rhizo) Mucor miehei | ++ | No secondary products |
| | | Monoglyceride accumlation |
| | | (24 h) |
| Pancreatin | o | |
| Penicillium roquefortii | ++ | No secondary products |
| | | Monoglyceride accumlation |
| | | (24 h) |
| Rhizopus arrhizus | ++ | No secondary products |
| | | Monoglyceride accumulation |
| | | (24 h) |
| | | Degree of hydrolysis |
| | | >85% after 48 h |
| Rhizopus niveus | ++ | No secondary products |
| | | Monoglyceride accumulation |
| | | (24 h) |
| Thermomyces lanugenosus | ++++ | No secondary products |
| (Lipozym TL 100 I, kosher, | | Monoglyceride accumulation |
| | | (24 h) |
| food grade) | | >85% after 72 h |
| Thermomyces lanugenosus | ++++ | No secondary products |
| (lipolase, detergent quality) | | Monoglyceide accumulation |
| | | (24 h) |
| | | >85% after 72 h |

### Example 2

### Investigation of Lipase Combinations for the Complete Hydrolysis of Castor Oil

7 mixtures each containing 5 castor oil and 5 g dist. water were stirred at 25° C. to form an emulsion. Quantities of 10 μl Thermomyces lanugenosus lipase (Lipozym TL 100 l) were pipetted into each mixture. A second lipase (10 μl of a 0.5% solution) was added to 6 of the mixtures, the seventh mixture serving as control. The emulsions were stirred for 36 h, separated by centrifuging and analyzed for hydrolysis activity by thin layer chromatography. The relative percentage of mono- and diglycerides in the reaction mixture was evaluated.

TABLE 2
Comparison of the hydrolysis activity of various lipase combinations

| | | Hydrolysis | Secondary |
|---|---|---|---|
| Lipase 1 | Lipase 2 | activity | products |
| Thermomyces lanugenosus | Penicillium | ++++ | None |
| | camembertii | | |
| Thermomyces lanugenosus | Rhizopus niveus | +++ | None |
| Thermomyces lanugenosus | Mucor javanicus | ++ | None |
| Thermomyces lanugenosus | Aspergillus niger | ++ | None |
| Thermomyces lanugenosus | Candida rugosa | +++ | Some |
| | | | formation |
| Thermomyces lanugenosus | Rhizopus oryzae | ++ | None |
| Thermomyces lanugenosus | — | ++ | None |

The combination of Thermomyces lanugenosus and Penicillium camembertii lipase is particularly preferred for the hydrolysis of castor oil because this lipase combination has a synergistic hydrolysis effect. Another preferred lipase is Rhizopus niveus in combination with Thermomyces lanugenosus.

### Example 3

### Optimization of Lipase Mixing Ratio for Hydrolysis of Castor Oil

Objective: the optimum mixing ratio of the enzymes to be determined using the particularly preferred lipase combination (Thermomyces lanugenosus+Penicillium camembertii) determined in Example 2.

Procedure: 5 mixtures each containing 25 g castor oil and 25 g distilled water were stirred at 25° C. to form an emulsion. Thermomyces lanugenosus solution (Lipozym TL, Novo Nordisk) and Penicillium camembertii (Lipase G, Amano) were then added in the following concentrations.

| Mixture | 1 | 2 | 3 | 4 | 5 |
|---|---|---|---|---|---|
| Thermomyces | 0.5 ml | — | 0.5 ml | 0.5 ml | 0.5 ml |
| lanugenosus lipase | | | | | |
| Penicillium | — | 20 mg | 5 mg | 20 mg | 80 mg |
| camembertii lipase | | | | | |

The emulsions were separated by centrifuging (5 mins. 13,000 r.p.m.) at various ratio times and analyzed for acid formation by gas chromatography.

TABLE 3
Formation of ricinoleic acid as a function of reaction time

| Reaction | Acid formation | | | | |
|---|---|---|---|---|---|
| time | Mixture 1 | Mixture 2 | Mixture 3 | Mixture 4 | Mixture 5 |
| 1 h | 17% | 1% | 16% | 13% | 10% |
| 4 h | 31% | 3% | 32% | 35% | 42% |
| 16 h | 57% | 2% | 64% | 77% | 77% |
| 30 h | 66% | 3% | 77% | 88% | 89% |
| 46 h | 70% | 3% | 83% | 91% | 92% |

The test shows that a ratio of Thernomyces lanugenosus lipase (Lipozym TL) to Penicillium camembertii lipase (Lipase G, Amano Pharmaceuticals) of about 25:1, based on the weighed sample of the commercially obtainable enzyme preparations, is a preferred enzyme ratio. An increase in the lipase G component increases the formation of free acid only negligibly whereas a reduction in the lipase G component leads to a reduction in the formation of free acid.

### Example 4

### Hydrolysis of Castor Oil by a Two-Stage Process

4,800 kg castor oil and 2,080 kg water were stirred at 30° C. to form an emulsion. 700 g lipase from Penicillium camembertii (Lipase G, Amano) and 14 kg lipase from Thermomyces lanugenosus (Lipozym TL, Novo Nordisk) were added with stirring. The mixture was stirred for 24 h at 30° C. The emulsion heated to 80° C. was then separated by gravity. The oil phase was re-stirred with 2,080 kg water at 30° C. to form an emulsion and 700 g lipase from Penicillium camembertii (Lipase G, Amano) and 14 kg lipase from Thermomyces lanugenosus (Lipozym TL, Novo Nordisk) were added. The mixture was reincubated with stirring for 24 h at 30° C., heated to 80° C. and separated by gravity separation.

After the first reaction stage, an 88% conversion of the castor oil was achieved with no formation of secondary products. In all, a more than 99% conversion of the castor oil was achieved with no secondary product formation.

The residual enzyme activity was well below 1% of the quantity of enzymes used. Composition of the end product according to GC analysis:

| | |
|---|---|
| acid: | 99.8% |
| monoglycerides: | 0.1% |
| diglycerides: | 0.1% |
| triglycerides: | 0% |

### Example 5

### Hydrogenation with Nickel Catalyst

500 ml ricinoleic acid from Example 3 were dried in vacuo and hydrogenated in a 500 ml autoclave for 1 h at 120° C./20 bar hydrogen pressure in the presence of 0.4% by weight catalyst (nickel catalyst Nysofact IQ 101). The ca. 100° C. hot product was filtered with acid-activated bleaching earth (10% by weight) and 1% by weight Trisyl 300 was added. After stirring for 20 mins. at 90° C. and drying, the mixture was separated in vacuo in a nutsch filter. The 12-hydroxystearic acid obtained has a melting range of 72-81° C.

Characteristics:

| | |
|---|---|
| OH value: | 159 |
| Iodine value: | 2.2 |
| Acid value: | 170 |

### Example 6

### Hydrogenation with Palladium

500 ml ricinoleic acid from Example 3 were dried in vacuo and hydrogenated in a 500 ml autoclave for 3 h at 90° C./150 bar hydrogen in the presence of 0.5% by weight catalyst (palladium/carbon catalyst: 5% palladium on active carbon [Norrit Pulver]). After hydrogenation, the product was freed from the catalyst by pressure filtration.

Characteristics:

| | |
|---|---|
| OH value: | 144 |
| Iodine value: | 4 |
| Acid value: | 173 |

1. A process for isolating 12-hydroxystearic acid and salts thereof from a native fat or oil, more particularly from castor oil, characterized in that
a) in a first step, the native fat or oil is hydrolyzed at a temperature of 15 to 50° C. in the presence of one or more enzymes as catalyst, ricinoleic acid being formed,
b) the glycerol formed and the enzyme are removed,
c) the hydrolyzate is catalytically hydrogenated,
d) the product thus obtained is made up into an end product.

2. A process as claimed in claim 1, characterized in that the enzymes used in step a) are selected from the group of hydrolases.

3. A process as claimed in claim 1 and/or 2, characterized in that the hydrolases are selected from the lipases from Aspergillus oryzae, Aspergillus niger, Bacillus species, Penicillium, camembertii, Pseudomonas cepacia, Candida lipolytica, Geotrichum candidum, Penicillium roqueforti, Rhizopus arrhizus, Rhizopus oryzae, Rhizomucor miehei, Rhizopus niveus, Mucorjavanicus and Thermomyces lanugenosus.

4. A process as claimed in any of claims 1 to 3, characterized in that the enzyme(s) is/are used in a quantity of 0.012 to 0.505% by weight, based on the total quantity of native fat or oil used.

5. A process as claimed in any of the preceding claims, characterized in that a buffer is used in a quantity of 0.01 to 0.2% by weight, based on the total quantity of native fat or oil, in step a) of the process.

6. A process as claimed in claim 5, characterized in that the buffer is a phosphate buffer.

7. A process as claimed in claim 1, characterized in that the separation process in step b) is centrifuging or phase separation by heating of the emulsion to 70-90° C.

8. A process as claimed in claim 1, characterized in that the hydrogenation catalysts in step c) are selected from the group consisting of Pt, Pd, Rh, Mo, W, Cr, Fe, Co, Al and Ni.

9. A process as claimed in claim 8, characterized in that the hydrogenation is carried out at a temperature of 70 to 150° C.

10. A process as claimed in claim 8, characterized in that the hydrogenation is carried out under a pressure of the hydrogen gas of 1 to 300 bar.

11. A process as claimed in claims 8 to 10, characterized in that the metal catalyst is used in a quantity of 0.2 to 5% by weight, based on the total quantity of fat or oil used.

12. A process as claimed in claim 1, characterized in that step c) is carried our by spray drying.
